(19) United States
(12) Patent Application Publication
(10) Pub. No.: US 2015/0119802 A1
(43) Pub. Date: Apr. 30, 2015

(54) PORTABLE VEIN LOCATING DEVICE

(71) Applicant: Constantin Dumitrescu, Calgary (CA)

(72) Inventor: Constantin Dumitrescu, Calgary (CA)

(21) Appl. No.: 14/065,405

(22) Filed: Oct. 28, 2013

(51) Int. Cl. A61M 5/42

(52) U.S. Cl. CPC A61M 5/427
USPC 604/116

(57) ABSTRACT

A vein locating device for locating a vein on a patient has a housing and an objective lens. A condenser lens is disposed within the housing and is aligned with the objective lens along a common axis. There is an infrared light source for emitting infrared light from the vein locating device onto an area of skin of the patient. There is a sensor circuit which includes a first photosensor, a second photosensor, a comparator, and a marking light.

### BACKGROUND OF THE INVENTION

1. Field of the Invention

The present invention relates to a vein locating device and, in particular, to a compact and portable vein locating device.

2. Description of the Related Art

It is known to provide vein locating devices to assist health care providers in locating a vein beneath the skin of a patient during venipuncture. Conventional vein locating devices however are relatively bulky and may be difficult to transport. There have accordingly been attempts to develop a compact and portable vein detector which may be easily transported by a health care provider.

An example of a compact and portable vein detector is disclosed in International Patent Publication Number WO 2006/014868, which was published on Feb. 9, 2006 in the name of Wu et al., and is directed to a portable vein locating device which includes an infrared light source for emitting infrared light onto an area of skin of a patient. A vein imaging module determines an intuitive location of a vein beneath the skin by detecting the absence of backscattering of infrared light. In particular, as the vein locating device is passed over the skin, infrared light from the infrared light source penetrates the skin and is absorbed by veins under the skin but backscattered by fat and other tissues surrounding the veins. Infrared light is backscattered from areas about the veins is detected by infrared light detectors of the vein imaging module. The vein imaging module also includes a display for displaying the intuitive location of a vein underneath the skin. The display may be a light-emitting diode array or a liquid crystal display.

However, while the vein locating device disclosed by Wu et al. is compact and portable, only an intuitive indication of a location of a vein relative to the vein detector is displayed by the vein imaging module. It is ultimately necessary for the health care provider to determine the location of the vein beneath the skin. There accordingly remains a need for an improved, compact and portable vein locating device.

### SUMMARY OF THE INVENTION

There is accordingly provided a vein locating device for locating a vein on a patient. The vein locating device has a housing and an objective lens. A condenser lens is disposed within the housing and is aligned with the objective lens along a common axis. There is an infrared light source for emitting infrared light from the vein locating device onto an area of skin of the patient. There is a sensor circuit which includes a first photosensor, a second photosensor, a comparator, and a marking light.

The first photosensor is positioned to sense infrared light backscattered from a central portion of the area of skin. The second photosensor is positioned to sense infrared light backscattered from a peripheral portion of the area of skin. The first photosensor generates a signal that is proportional to an intensity of infrared light sensed. The second photosensor generates a signal that is proportional to an intensity of infrared light sensed.

The comparator generates a signal when the signal generated by the first photosensor differs from the signal generated by the second photosensor. The signal generated by the comparator triggers the marking light to mark the central portion of the area of skin with visible light to indicate a location of a vein.

The housing of the vein locating device may be elongate. The objective lens and the condenser lens may be aligned along a longitudinal axis of the housing. The objective lens may be a biconvex lens. The condenser lens may be a plano-convex lens and a plane side of the plano-convex lens may face the objective lens. The first photosensor may be disposed between the objective lens and the condenser lens. The first photosensor and the second photosensor may be on opposite sides of the condenser lens.

The comparator may be a differential amplifier. The signal generated by the first photosensor may be a voltage signal received by the differential amplifier. The signal generated by the second photosensor may be a voltage signal received by the differential amplifier. There may be an operational amplifier connected in series between the first photosensor and the differential amplifier. There may be an operational amplifier connected in series between the second photosensor and the differential amplifier.

The marking light may be a visible spectrum light emitting-diode disposed adjacent to the first photosensor. The infrared light source may be an infrared light-emitting diode disposed about the objective lens.

### BRIEF DESCRIPTIONS OF DRAWINGS

The invention will be more readily understood from the following description of the embodiments thereof given, by way of example only, with reference to the accompanying drawings, in which:

FIG. 1 is a perspective view of an improved portable vein locating device;

FIG. 2 is an end view of a head of the portable vein locating device of FIG. 1;

FIG. 3 is a simplified schematic of the portable vein locating device of FIG. 1;

FIG. 4 is a simplified circuit diagram of the portable vein locating device of FIG. 1; and

FIG. 5 is a perspective view showing use of the portable vein locating device of FIG. 1 in use with fragments of the hands and arms of a health care provider and patient.

### DESCRIPTIONS OF THE PREFERRED EMBODIMENTS

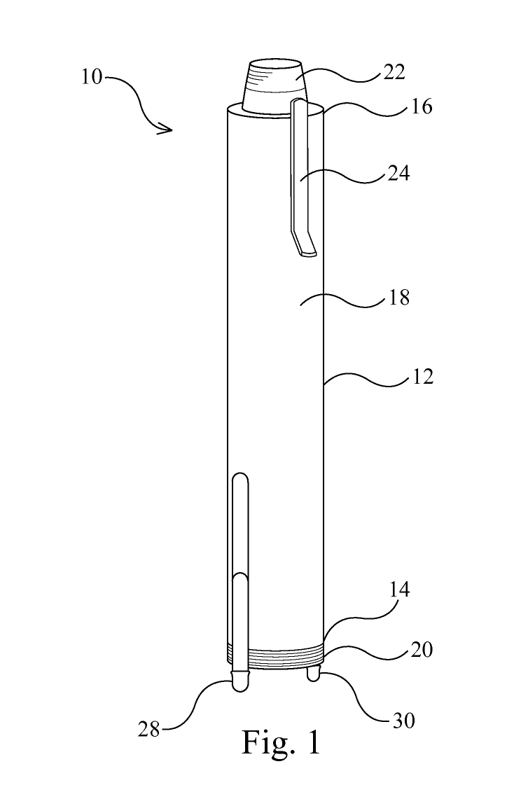

Referring to the drawings and first to FIG. 1, an improved vein locating device 10 is shown. The vein locating device 10 comprises a housing 12 which is elongate, in this example, but may be any suitable shape. The housing 12 has a first end 14 and a second end 16 which is opposite of the first end 14. There is a cylindrical outer housing wall 18 extending between the first end 14 of the housing 12 and the second end 16 of the housing 12. A head 20 of the vein locating device 10 is disposed at the first end 14 of the housing 12. A switch 22 of the vein locating device 10 is disposed at the second end 16 of the housing 12. In this example, the switch 22 is a biased switch in the form of a push-button switch. However, in other examples, the switch may be a toggle switch disposed on the cylindrical outer wall of the housing or a rotary switch integrated with the housing. The switch 22 is actuated by a user to turn the vein locating device 10 ON and OFF. There is a clip 24 mounted on the cylindrical outer housing wall 18 but this is not strictly required. The clip 24 may be used to secure the vein locating device 10 to clothing or another article.

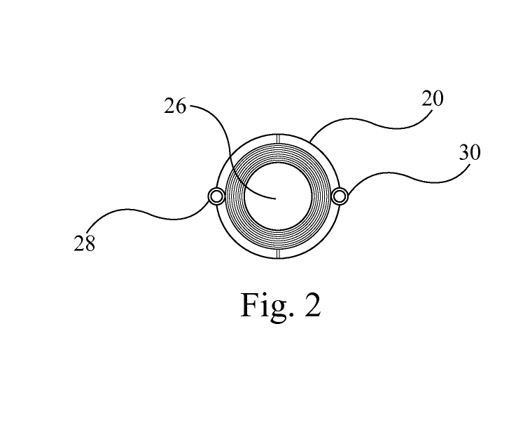

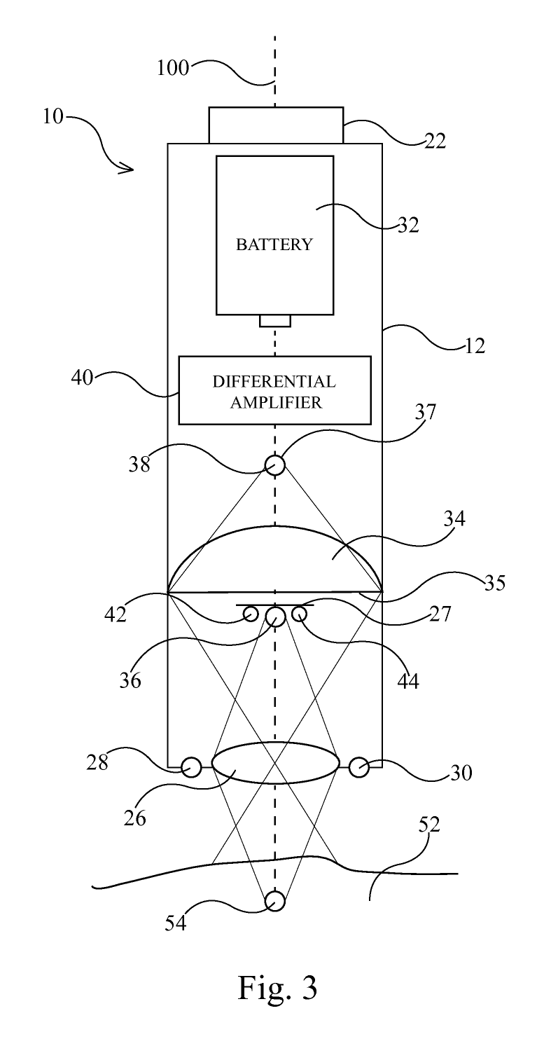

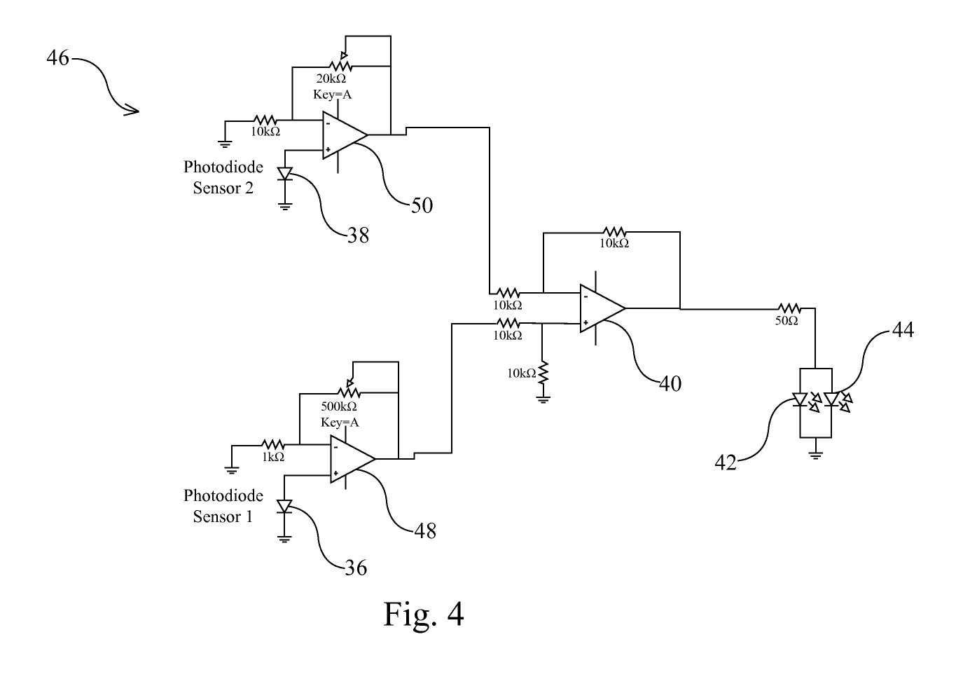

The head 20 of the vein locating device 10, which is best shown in FIG. 2, includes an objective lens in the form of a biconvex lens 26 and an infrared light source in the form of infrared light-emitting diodes 28 and 30 which are circumferentially spaced-apart about the biconvex lens 26. The biconvex lens 26 allows light to travel into and out of the housing 12. A power supply in the form of a battery 32, which is shown in FIG. 3, supplies power to the infrared light-emitting diodes 28 and 30. Referring now specifically to FIG. 3, there is a condenser lens in the form of a plano-convex lens 34 disposed in the housing 12. The biconvex lens 26 and the plano-convex lens 34 are aligned along a longitudinal axis 100 of the vein detector 10 but may be aligned along any common axis. A first photosensor 36, a second photosensor 38, a comparator in the form of a differential amplifier 40, and a marking light in the form of light-emitting diodes 42 and 44 are also disposed within the housing 12 of the vein detector 10, and are part of a sensor circuit 46 which is shown in FIG. 4.

The first photosensor 36 is an infrared-sensitive photodiode but may be any infrared-sensitive photosensor, for example, a phototransistor or a photovoltaic cell. The anode of the first photosensor 36 is electrically connected to a positive input terminal of a first operational amplifier 48. The first operational amplifier 48 amplifies a voltage signal generated by the first photosensor 36 when the first photosensor 36 senses infrared light. The strength of the voltage signal generated by the first photosensor 36 is proportional to the intensity of the infrared light sensed. The first photosensor 36 may be shielded from ambient light.

The second photosensor 38 is also an infrared-sensitive photodiode but may likewise be any infrared-sensitive photosensor. The anode of the second photosensor 38 is electrically connected to a positive input terminal of a second operational amplifier 50. The second operational amplifier 50 amplifies a voltage signal generated by the second photosensor 38 when the second photosensor 38 senses infrared light. The strength of the voltage signal generated by the second photosensor 38 is proportional to the intensity of the infrared light sensed. The second photosensor 38 may be shielded from ambient light.

The amplification of the signal generated by the first photosensor 36 is greater than the amplification of the signal generated by the second photosensor 38. This is because the area being sensed by the first photosensor 36 is smaller than the area being sensed by the second photsensor 38, as will be discussed in greater detail below.

The operational amplifiers 48 and 50 are high impedance amplifiers and are connected in parallel to the differential amplifier 40. An output of the first operational amplifier 48 is electrically connected to a positive input terminal of the differential amplifier 40 while an output of the second operational amplifier 50 is electrically connected to a negative input terminal of the differential amplifier 40. An output of the differential amplifier 40 is connected in parallel to anodes of marking light-emitting diodes 42 and 44. The differential amplifier 40 generates a voltage signal when there is a difference in the voltage signals generated by the first photosensor 36 and the second photosensor 38. The voltage signal generated by the differential amplifier 40 triggers the marking light-emitting diodes 42 and 44 to light up and emit light though the head 20 of the vein locating device 10. The marking light-emitting diodes 42 and 44 are blue light-emitting diodes in this example but may be any light sources that emit light in the visible spectrum.

Referring back to FIG. 3, the relative positionings of the plano-convex lens 34, the first photosensor 36, the second photosensor 38, and the marking light-emitting diodes 42 and 44 within the housing 12 are shown. A plane side of the plano-convex lens 34 faces the biconvex lens 26 and, in this example, the first photosensor 36 and the second photosensor 38 are on opposite sides of the plano-convex lens 34 with the first photosensor 36 being disposed between the biconvex lens 26 and the plano-convex lens 34. The first photosensor 36 is positioned at a focal point 27 of the biconvex lens 26 within the housing 12. The second photosensor 38 may preferably be positioned at a focal point 37 of the plano-convex lens 34 within the housing 12. The marking light-emitting diodes 42 and 44 are disposed on opposite sides of the first photosensor 36.

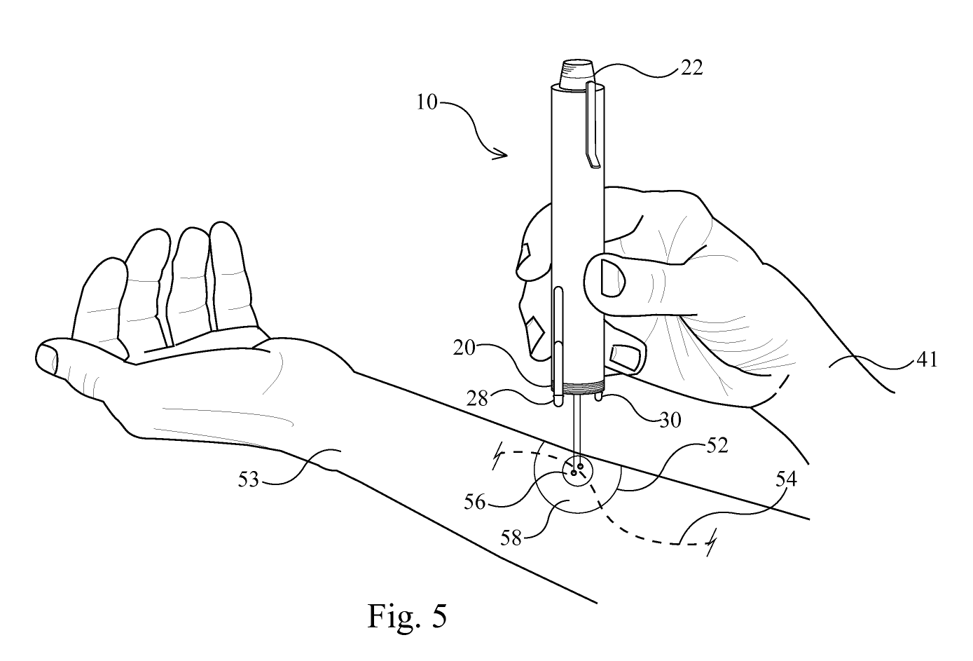

In operation, and with reference to FIGS. 3 and 5, a health care provider 41 positions the vein locating device 10 so that the head 20 faces an area of skin 52 of a patient being subject to venipuncture. The vein locating device 10 is turned ON by actuating the switch 22 which causes the infrared light-emitting diodes 28 and 30 to emit infrared light onto the area of skin 52 of a patient 53. The infrared light emitted by the light-emitting diodes 28 and 30 will generally be absorbed by veins under the skin but is backscattered by surrounding fat and tissue. Accordingly, when infrared light from infrared light-emitting diodes 28 and 30 is emitted onto an area of skin beneath which there is no vein, the first photosensor 36 and the second photosensor 38 senses the same infrared light intensity on the skin and both generate equal voltage signals. This is because there is no absorption of the infrared light emitted onto an area of skin beneath which there is no vein.

However, when infrared light from the vein locating device 10 is emitted onto an area of skin beneath which there is a vein, the first photosensor 36 and the second photosensor 38 may sense different intensities of infrared light on the skin and generate different voltage signals. As best shown in FIG. 5, the first photosensor 36 senses infrared light backscattered from a central area 56 of the area of skin 52 that is subject to infrared light while the second photosensor 38 senses infrared light backscattered from a peripheral area 58 of the area of skin 52 that is also subject to infrared light. The peripheral area 58 of the area of skin 52 extends about the central area 56 of the area of skin 52 and is larger than the central area 56 of the area of skin 52. For example, the central area 56 of the area of skin 52 may be 1 mm² while the peripheral area 58 of the area of skin 52 may be between 100 mm² and 300 mm².

The intensity of infrared light sensed by the first photosensor 36 will be less than the intensity of infrared light sensed by the second photosensor 38 when there is a vein 54 located under the central area 56 of the area of skin 52. This is because the central area 56 of the area of skin 52 is smaller than the peripheral area 58 of the area of skin 52, and the vein 54 extends under a proportionally much larger portion of the central area 56 of the area of skin 52 as compared to the peripheral area 58 of the area of skin 52. Accordingly, a proportionally larger amount of the infrared light will be absorbed by the vein 54 in the central area 56 of the area of skin 52 as compared to the peripheral area 58 of the area of skin 52. The voltage signals generated by the first photosensor 36 and the second photosensor 38 will therefore be different because the voltage signals generated are proportional to the intensity of the infrared light backscatter sensed. The difference in voltage signals results in the differential amplifier 40 generating a voltage signal which causes the marking light-emitting diodes 42 and 44 to light up and emit visible light to mark a location of the vein 54 on the skin 52.

It will be understood by a person skilled in the art that many of the details provided above are by way of example only, and are not intended to limit the scope of the invention which is to be determined with reference to the following claims.

What is claimed is:

1. A vein locating device, for locating a vein on a patient, the vein locating device comprising:
a housing;
an objective lens;
a condenser lens disposed within the housing, the condenser lens being aligned with the objective lens along a common axis;
an infrared light source for emitting infrared light from the vein locating device onto an area of skin of the patient; and
a sensor circuit including a first photosensor, a second photosensor, a comparator, and a marking light;
wherein the first photosensor is positioned to sense infrared light backscattered from a central portion of the area of skin and the second photosensor is positioned to sense infrared light backscattered from a peripheral portion of the area of skin, the first photosensor generating a signal that is proportional to an intensity of infrared light sensed and the second photosensor generating a signal that is proportional to an intensity of infrared light sensed, the comparator generating a signal when the signal generated by the first photosensor differs from the signal generated by the second photosensor, and the signal generated by the comparator causing the marking light to mark the central portion of the area of skin with visible light to indicate a location of a vein.

2. The vein locating device as claimed in claim 1 wherein the housing is elongate, and the objective lens and the condenser lens are aligned along a longitudinal axis of the housing.

3. The vein locating device as claimed in claim 1 wherein the objective lens is a biconvex lens.

4. The vein locating device as claimed in claim 1 wherein the condenser lens is a plano-convex lens and a plane side of the plano-convex lens faces the objective lens.

5. The vein locating device as claimed in claim 1 wherein the first photosensor is disposed between the objective lens and the condenser lens, and the first photosensor and the second photosensor are on opposite sides of the condenser lens.

6. The vein locating device as claimed in claim 1 wherein:
the comparator is a differential amplifier;
the signal generated by the first photosensor is a voltage signal received by the differential amplifier; and
the signal generated by the second photosensor is a voltage signal received by the differential amplifier.

7. The vein locating device as claimed in claim 6 further including an operational amplifier connected in series between the first photosensor and the differential amplifier.

8. The vein locating device as claimed in claim 6 further including an operational amplifier connected in series between the second photosensor and the differential amplifier.

9. The vein locating device as claimed in claim 1 wherein the marking light is a visible spectrum light emitting-diode disposed adjacent to the first photosensor.

10. The vein locating device as claimed in claim 1 wherein the infrared light source is an infrared light-emitting diode disposed about the objective lens.
